(19) United States
(12) Patent Application Publication
(10) Pub. No.: US 2012/0245638 A1
(43) Pub. Date: Sep. 27, 2012

(54) SACRAL BRACE

(75) Inventor: Calin Druma, San Jose, CA (US)

(73) Assignee: KYPHON SÀRL, Neuchatel (CH)

(21) Appl. No.: 13/053,944

(22) Filed: Mar. 22, 2011

(51) Int. Cl. A61B 17/70

(52) U.S. Cl. 606/248

(57) ABSTRACT

Medical devices for the treatment of spinal conditions are described herein. The medical device includes a support portion that is adapted to be fixed to the sacrum and a bumper that is adapted to be located adjacent to the inferior portion of the spinous process of the L5 vertebra. The medical device acts as a spacer with respect to the L5 vertebra and the sacrum to maintain distraction therebetween when the spine moves in extension. The medical device may also include a superior portion adapted to extend over the superior portion of the superior spinous process to control flexion of the spine.

### BACKGROUND

This invention relates generally to devices for the treatment of spinal conditions, and more particularly, to the treatment of various spinal conditions that cause back pain. Even more particularly, this invention relates to devices that may be placed between adjacent spinous processes to treat various spinal conditions. For example, spinal conditions that may be treated with these devices may include spinal stenosis, degenerative disc disease (DDD), disc herniations and spinal instability, among others.

The clinical syndrome of neurogenic intermittent claudication due to lumbar spinal stenosis is a frequent source of pain in the lower back and extremities, leading to impaired walking, and causing other forms of disability in the elderly. Although the incidence and prevalence of symptomatic lumbar spinal stenosis have not been established, this condition is the most frequent indication of spinal surgery in patients older than 65 years of age.

Lumbar spinal stenosis is a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop lumbar spinal stenosis each year. For patients who seek the aid of a physician for back pain, approximately 12%-15% are diagnosed as having lumbar spinal stenosis.

Common treatments for lumbar spinal stenosis include physical therapy (including changes in posture), medication, and occasionally surgery. Changes in posture and physical therapy may be effective in flexing the spine to decompress and enlarge the space available to the spinal cord and nerves—thus relieving pressure on pinched nerves. Medications such as NSAIDS and other anti-inflammatory medications are often used to alleviate pain, although they are not typically effective at addressing spinal compression, which is the cause of the pain.

Surgical treatments are more aggressive than medication or physical therapy, and in appropriate cases surgery may be the best way to achieve lessening of the symptoms of lumbar spinal stenosis and other spinal conditions. The principal goal of surgery to treat lumbar spinal stenosis is to decompress the central spinal canal and the neural foramina, creating more space and eliminating pressure on the spinal nerve roots. The most common surgery for treatment of lumbar spinal stenosis is direct decompression via a laminectomy and partial facetectomy. In this procedure, the patient is given a general anesthesia and an incision is made in the patient to access the spine. The lamina of one or more vertebrae may be partially or completely removed to create more space for the nerves. The success rate of decompressive laminectomy has been reported to be in excess of 65%. A significant reduction of the symptoms of lumbar spinal stenosis is also achieved in many of these cases.

The failures associated with a decompressive laminectomy may be related to postoperative latrogenic spinal instability. To limit the effect of latrogenic instability, fixation and fusion may also be performed in association with the decompression. In such a case, the intervertebral disc may be removed, and the adjacent vertebrae may be fused. A discectomy may also be performed to treat DDD and disc herniations. In such a case, a spinal fusion would be required to treat the resulting vertebral instability. Spinal fusion is also traditionally accepted as the standard surgical treatment for lumbar instability. However, spinal fusion sacrifices normal spinal motion and may result in increased surgical complications. It is also believed that fusion to treat various spinal conditions may increase the biomechanical stresses imposed on the adjacent segments. The resultant altered kinematics at the adjacent segments may lead to accelerated degeneration of these segments.

As an alternative or complement to the surgical treatments described above, an interspinous process device may be implanted between adjacent spinous processes of adjacent vertebrae. The purposes of these devices are to provide stabilization after decompression, to restore foraminal height, and to unload the facet joints. They may also allow for the preservation of a range of motion in the adjacent vertebral segments, thus avoiding or limiting possible overloading and early degeneration of the adjacent segments as induced by fusion. The vertebrae may or may not be distracted before the device is implanted therebetween. An example of such a device is the interspinous prosthesis described in U.S. Pat. No. 6,626,944, the entire contents of which are expressly incorporated herein by reference. This device, commercially known as the DIAM® spinal stabilization system, is designed to restabilize the vertebral segments as a result of various surgical procedures or as a treatment of various spinal conditions. It limits extension and may act as a shock absorber, since it provides compressibility between the adjacent vertebrae, to decrease intradiscal pressure and reduce abnormal segmental motion and alignment. This device provides stability in all directions and maintains the desired separation between the vertebral segments all while allowing motion in the treated segment.

Although currently available interspinous process devices typically work for their intended purposes, they could be improved. For example, where the spacer portion of the implant is formed from a hard material to maintain distraction between adjacent vertebrae, point loading of the spinous process can occur due to the high concentration of stresses at the point where the hard material of the spacer contacts the spinous process. This may result in excessive subsidence of the spacer into the spinous process. In addition, if the spinous process is osteoporotic, there is a risk that the spinous process could fracture when the spine is in extension. In addition, because of the human anatomy and the complex biomechanics of the spine, some currently available interspinous process devices may not be easily implantable in certain locations in the spine.

The spine is divided into regions that include the cervical, thoracic, lumbar, and sacrococcygeal regions. The cervical region includes the top seven vertebrae identified as C1-C7. The thoracic region includes the next twelve vertebrae identified as T1-T12. The lumbar region includes five vertebrae L1-L5. The sacrococcygeal region includes five fused vertebrae comprising the sacrum. These five fused vertebrae are identified as the S1-S5 vertebrae. Four or five rudimentary members form the coccyx.

The sacrum is shaped like an inverted triangle with the base at the top. The sacrum acts as a wedge between the two iliac bones of the pelvis and transmits the axial loading forces of the spine to the pelvis and lower extremities. The sacrum is rotated anteriorly with the superior endplate of the first sacral vertebra angled from about 30 degrees to about 60 degrees in the horizontal plane. The S1 vertebra includes a spinous process aligned along a ridge called the medial sacral crest. However, the spinous process on the S1 vertebrae may not be well defined, or may be non-existent, and therefore may not be adequate for supporting an interspinous process device positioned between the L5 and S1 spinous processes.

Thus, a need exists for an interspinous process device that may be readily positioned between the L5 and S1 spinous processes. Moreover, there is a need to provide an interspinous process device that can provide dynamic stabilization to the instrumented motion segment and not affect adjacent segment kinematics.

### SUMMARY

A spinal implant is described herein that is particularly adapted for placement in the area of the spinous processes of the L5 vertebra and the S1 vertebra. The implant includes a pair of support legs, each including a fixation portion along an inferior portion of the implant to fix the inferior portion of the implant to the S1 vertebra. The implant also includes a bumper portion connected to the support legs which are adapted to be fixed to the S1 vertebra with the bumper disposed adjacent to an inferior portion of the L5 spinous process when the implant is fixed to the S1 vertebra. The bumper controls relative motion in the L5-S1 vertebral segment. If desired, the support legs may include a superior portion, or the implant may include a superior tether portion, that is adapted to extend over the superior portion of the superior spinous process to control the amount of flexion of the vertebral segments when the implant is located in position in the patient's anatomy. The superior portion of the support legs may also be removable so it can be affixed to the implant after the implant is fixed to the inferior vertebra thereby facilitating implantation of the implant.

The fixation portions of the implant allow fixation devices, such as cortical screws, to extend through the fixation portions and into the pedicles of the S1 vertebra. In addition, the fixation portions define a path for the fixation device that has a compound angle with respect to the longitudinal axis of the implant. This compound angle for the fixation device facilitates fixation of the implant to the pedicles of the S1 vertebra by providing an appropriate trajectory for the fixation devices to engage the S1 pedicles. For example, each of the fixation portions may define a passage therethrough that extends at an angle of about 60 degrees away from the sagittal plane toward the rear of the implant and at an angle of about 5 degrees toward the top of the implant in a direction from the front of the implant toward the rear of the implant.

The support legs of the spinal implant described herein may be formed as a unitary body of a rigid material such as a titanium alloy. Alternatively, the material may have some inherent flexibility. The bumper portion of the spinal implant described herein may be formed of a flexible, elastic or compressible material such as a silicone elastomer that is more flexible than the support legs.

### BRIEF DESCRIPTION OF THE DRAWINGS

FIG. 1 is a front perspective view of one embodiment of a spinal implant;

FIG. 2 is a front elevation view of the embodiment of the spinal implant shown in FIG. 1;

FIG. 3 is a bottom plan view of the embodiment of the spinal implant shown in FIG. 1;

FIG. 4 is a front elevation view of the spinal implant shown in FIG. 1 mounted on a spine;

FIG. 5 is a side elevation view of the spinal implant shown in FIG. 1 mounted on a spine;

FIG. 6 is front perspective view showing an alternate embodiment of a spinal implant;

FIG. 7 is a front elevation view of the alternate embodiment of the spinal implant shown in FIG. 6;

FIG. 7A is a front elevation view of a variation of the alternate embodiment of the spinal implant shown in FIG. 6;

FIG. 8 is a bottom plan view of the alternate embodiment of the spinal implant shown in FIG. 6;

FIG. 9 is a side elevation view of the alternate embodiment of the spinal implant shown in FIG. 6;

FIG. 9A is a side elevation view of the variation of the alternate embodiment of the spinal implant shown in FIG. 7A;

FIG. 10 is a side elevation view of the alternate embodiment of the spinal implant shown in FIG. 6 mounted on a spine;

FIG. 11 is a front elevation view of the alternate embodiment of the spinal implant shown in FIG. 6 mounted on a spine;

FIG. 12 is front elevation view of yet another embodiment of a spinal implant;

FIG. 13 is a cross-sectional view of the embodiment of the spinal implant shown in FIG. 12 taken along line XIII-XIII in FIG. 12;

FIG. 14 is front elevation view of still another embodiment of a spinal implant;

FIG. 15 is a cross-sectional view of the embodiment of the spinal implant shown in FIG. 14 taken along line XV-XV in FIG. 14;

FIG. 16A is front elevation view of a further embodiment of a spinal implant with the superior portion removed;

FIG. 16B is a front elevation view showing the embodiment of the spinal implant shown in FIG. 16A with the superior portion fixed to the inferior portion;

FIG. 17A is front elevation view of a still further embodiment of a spinal implant with a flexible tether having its free ends unconnected to allow it to be easily wrapped over a superior spinous process; and

FIG. 17B is front elevation view showing the embodiment of the spinal implant shown in FIG. 17A with the free ends of the tether connected together;

### DETAILED DESCRIPTION

As used in this specification and the appended claims, the singular forms “a,” “an” and “the” include plural referents unless the context clearly dictates otherwise. Thus, for example, the term “a member” is intended to mean a single member or a combination of members, and “a material” is intended to mean one or more materials, or a combination thereof. Furthermore, the words “proximal” and “distal” refer to directions closer to and away from, respectively, an operator (e.g., surgeon, physician, nurse, technician, etc.) who would insert the medical device into the patient, with the tip-end (i.e., distal end) of the device inserted inside a patient's body first. Thus, for example, the device end first inserted inside the patient's body would be the distal end of the device, while the device end last to enter the patient's body would be the proximal end of the device.

As used in this specification and the appended claims, the terms “upper”, “top”, “lower”, “bottom”, “front”, “back”, “rear”, “left”, “right”, “side”, “middle” and “center”, and the like, refer to portions of or positions on the implant when the implant is oriented in its implanted position in a patient's anatomy.

As used in this specification and the appended claims, the term “axial plane” when used in connection with particular relationships between various parts of the implant means a plane that divides the implant into upper and lower parts. As shown in the FIGS., the axial plane is defined by the X axis and the Z axis. As used in this specification and the appended claims, the term “coronal plane” when used in connection with particular relationships between various parts of the implant means a plane that divides the implant into front and back parts. As shown in the FIGS., the coronal plane is defined by the X axis and the Y axis. As used in this specification and the appended claims, the term “sagittal plane” when used in connection with particular relationships between various parts of the implant means a plane that divides the implant into left and right parts. As shown in the FIGS., the sagittal plane is defined by the Y axis and the Z axis.

As used in this specification and the appended claims, the term “body” when used in connection with the location where the device of this invention is to be placed to treat spinal disorders, or to teach or practice implantation methods for the device, means a mammalian body. For example, a body can be a patient's body, or a cadaver, or a portion of a patient's body or a portion of a cadaver or a model of a patient's anatomy.

As used in this specification and the appended claims, the term “parallel” describes a relationship, given normal manufacturing or measurement or similar tolerances, between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

As used in this specification and the appended claims, the terms “normal”, “perpendicular” and “orthogonal” describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal, perpendicular or orthogonal to a curved surface when the line and the curved surface intersect at an angle of approximately 90 degrees within a plane. Two geometric constructions are described herein as being “normal”, “perpendicular”, “orthogonal” or “substantially normal”, “substantially perpendicular”, “substantially orthogonal” to each other when they are nominally 90 degrees to each other, such as for example, when they are 90 degrees to each other within a tolerance. Such tolerances can include, for example, manufacturing tolerances, measurement tolerances or the like.

A spinal implant is described herein that is particularly adapted for placement in the area of the spinous processes of the L5 vertebra and the S1 vertebra. However, it is to be understood that even though the following description of the implant is provided with reference to the L5 spinous process and the S1 spinous process, the implant may be used in the area of the other adjacent spinous process and the discussion of the L5 spinous process may be interpreted to include any superior spinous process and the S1 spinous process may be interpreted to include the adjacent inferior spinous process.

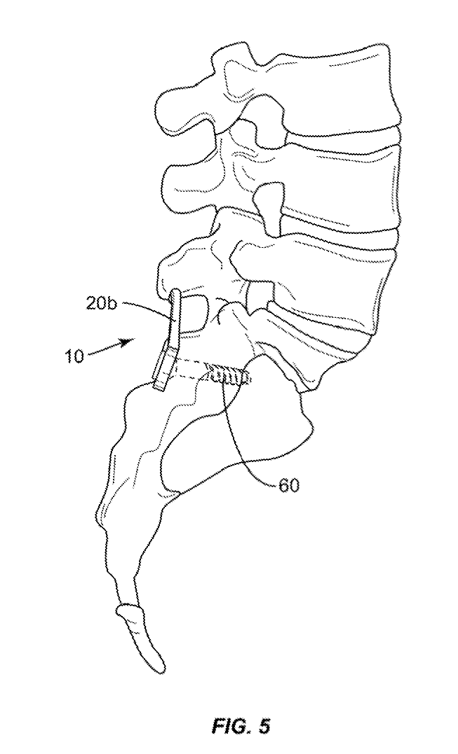

Implant 10 includes a bumper portion 50 and support legs 20a and 20b, which includes a pair of fixation portions 21a and 21b located at the inferior end of support legs 20a and 20b, respectively. Fixation portions 21a and 21b are adapted to allow implant 10 to be fixed in the desired location in the patient's anatomy. Each fixation portion may have a generally planar front and rear face defining a passage 22a and 22b, respectively, extending therethrough. To facilitate the implantation of implant 10 in the area of the L5 vertebra and the S1 vertebra, fixation portions 21a and 21b should be oriented at an angle to support legs 20a and 20b respectively to take into account the 30 degree to 60 degree sacral tilt. For example, fixation portions 21a and 21b may be oriented rearwardly at an angle of between about 20 degrees and about 40 degrees from support legs 20a and 20b. See, e.g. FIG. 5. A fixation device 60, such as a cortical screw, or similar device, may be passed through fixation portions 21a and 21b through passages 22a and 22b and thus fix implant 10 in the desired location on the spine. As such, the internal diameter of passages 22a and 22b should be sufficiently large to allow passage of fixation device 60 therethrough, but should not be so large as to allow too much “play”, or too big of a gap, between the outer surface of fixation device 60 and the walls defining passages 22a and 22b. For example, passages 22a and 22b may have an internal diameter that is about 0.5 mm to about 1 mm greater than the outer diameter of fixation device 60.

Passages 22a and 22b are oriented to provide fixation device 60 with the proper trajectory to engage the pedicles of the S1 vertebra when bumper portion 50 engages the inferior portion of the L5 spinous process. For example, the longitudinal axes of passages 22a and 22b may flare outwardly from about the mid-line of implant 10 and upwardly toward the top of implant 10. In particular, the longitudinal axes of passages 22a and 22b may extend at an angle of between about 45 degrees and about 60 degrees away from the sagittal plane toward the rear of implant 10 and at an angle of between about 5 degrees and 10 degrees toward the top of implant 10 in a direction from the front of implant 10 toward the rear of implant 10. See, e.g. FIG. 3.

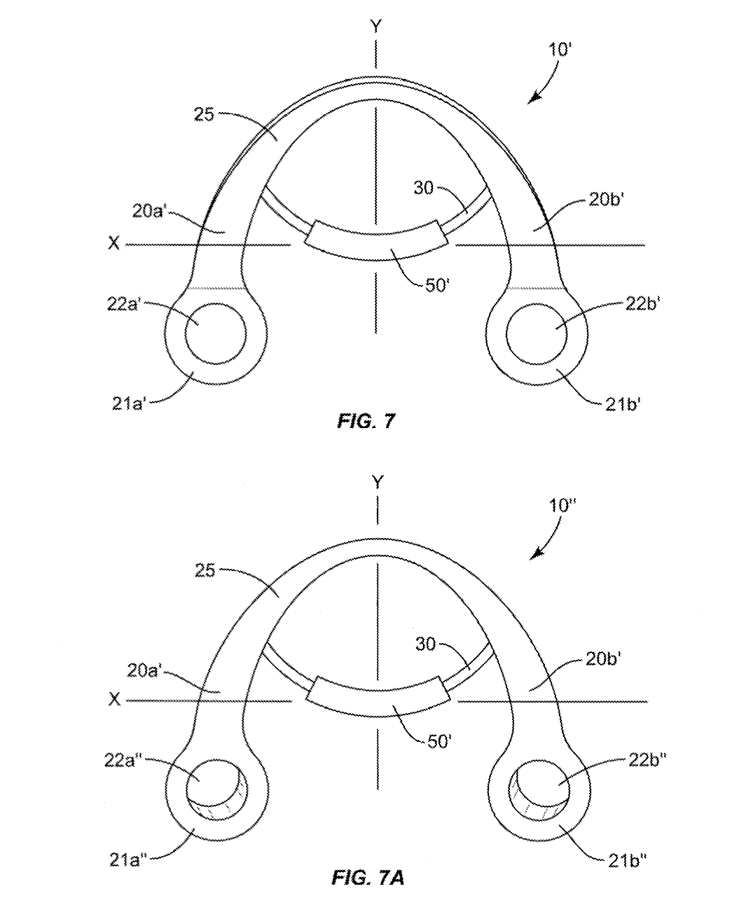

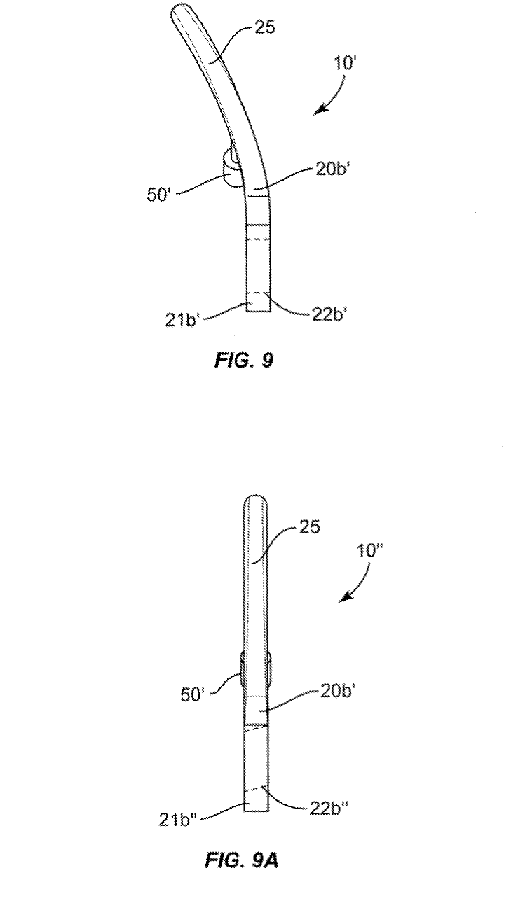

In an alternate embodiment, fixation portions 21a″ and 21b″ may each have a generally flat posterior face and a generally flat anterior face that are arranged generally parallel to each other and parallel to the posterior and anterior faces of support legs 20a′ and 20b′ respectively. See FIGS. 7A and 9A. In this embodiment, implant 10″ includes fixation portions 21a″ and 21b″ that are not disposed at an angle to support legs 20a′ and 20b′ respectively. Instead, the longitudinal axes of passages 22a′ and 22b′ may be oriented at appropriate angles to the posterior faces and anterior faces of fixation portions 21a″ and 21b″ to ensure that the trajectory of fixation device 60 therethrough allows them to engage the S1 pedicles, taking into account the sacral tilt, the orientation of fixation portions 21a″ and 21b″ to support legs 20a′ and 20b′ and the location of the pedicles.

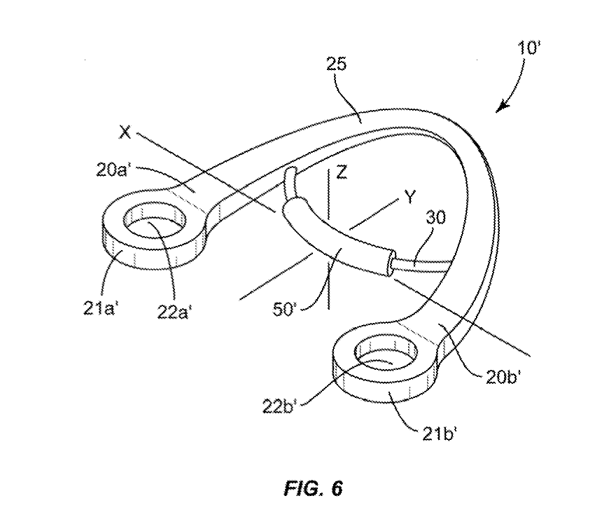

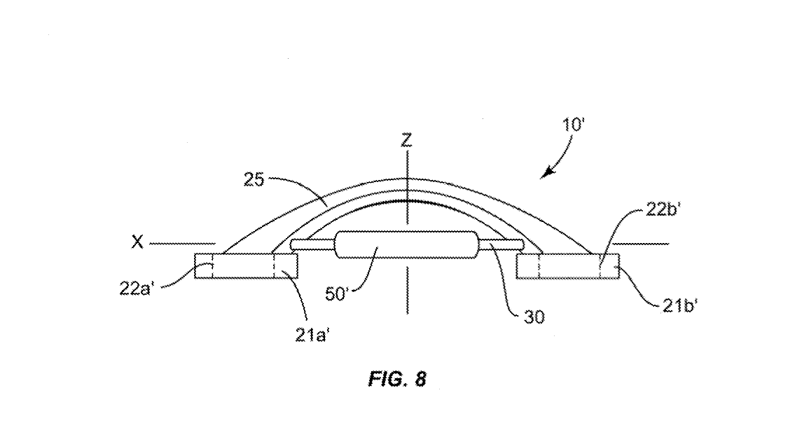

In an alternate embodiment, the longitudinal axes of passages 22a′ and 22b′ may be oriented perpendicular to the posterior faces and the anterior faces of fixation portions 21a′ and 21b′. See FIGS. 6, 7, 8, 9 and 10. With this configuration, the posterior and anterior faces of fixation portions 21a′ and 21b′ may be oriented at an angle to support legs 20a′ and 20b′ to allow the longitudinal axes of passages 22a′ and 22b′ to extend at the desired angles with respect to implant 10′. Thus, the posterior and anterior faces of fixation portions 21a′ and 21b′, and thus the longitudinal axes of passages 22a′ and 22b′, would take into account the sacral tilt, the orientation of fixation portions 21a′, 21b′ and support legs 20a′, 20b′ and the location of the pedicles to ensure that fixation devices 60 would have the appropriate trajectory to engage the S1 pedicles.

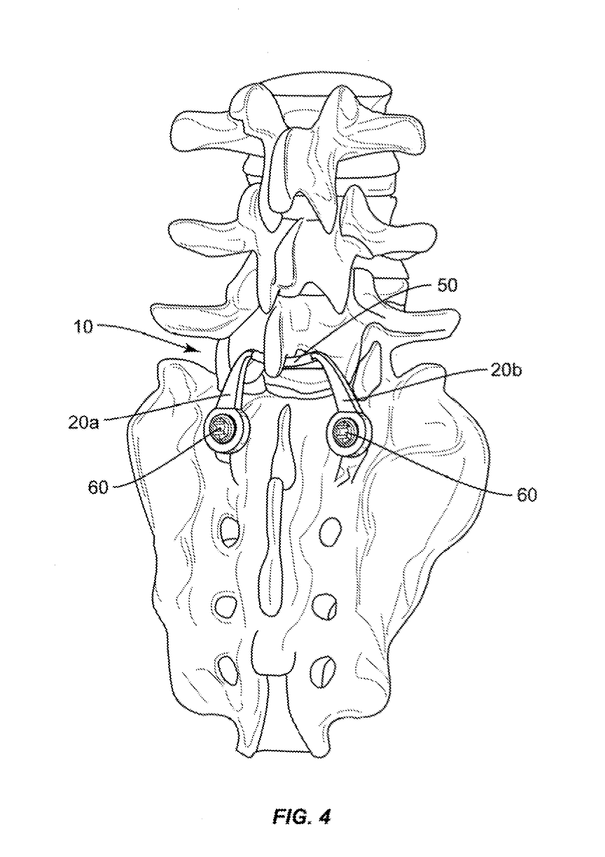

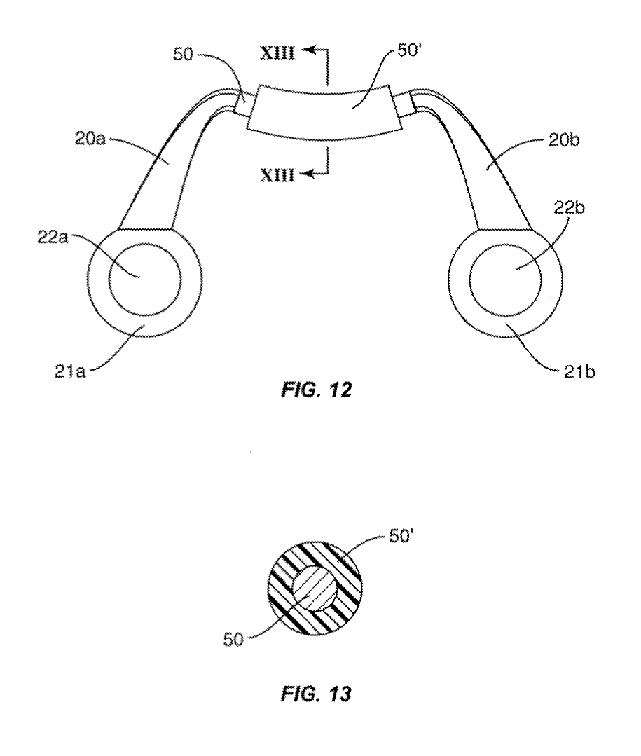

Implant 10 may be formed as a unitary body where support legs 20a and 20b and bumper portion 50 are formed from the same material, which may be substantially rigid or somewhat flexible. Alternatively, bumper portion 50 may be formed from a material such as a silicone elastomer having a durometer of between about 55A and about 85A that is more flexible, elastic or compressible than support legs 20a and 20b. Where bumper portion 50 is formed from a different material, bumper portion 50 may be connected to support legs 20a and 20b by any suitable connecting mechanism, such as an adhesive or mechanical connection. Bumper portion 50 is oriented such that it is located adjacent to a superior portion of each support leg 20a and 20b. This way, when implant 10 is properly located in the patient's anatomy with fixation portions 21a and 21b fixed to the sacrum, bumper portion 50 is located adjacent to an inferior portion of the L5 spinous process. See, e.g., FIG. 4. If desired, an additional bumper portion 50′ may be placed over bumper 50. See FIGS. 12 and 13. Bumper portion 50′ may have a hollow longitudinal passage to allow bumper portion 50′ to fit over bumper portion 50, which acts as a crossbar to support bumper portion 50′.

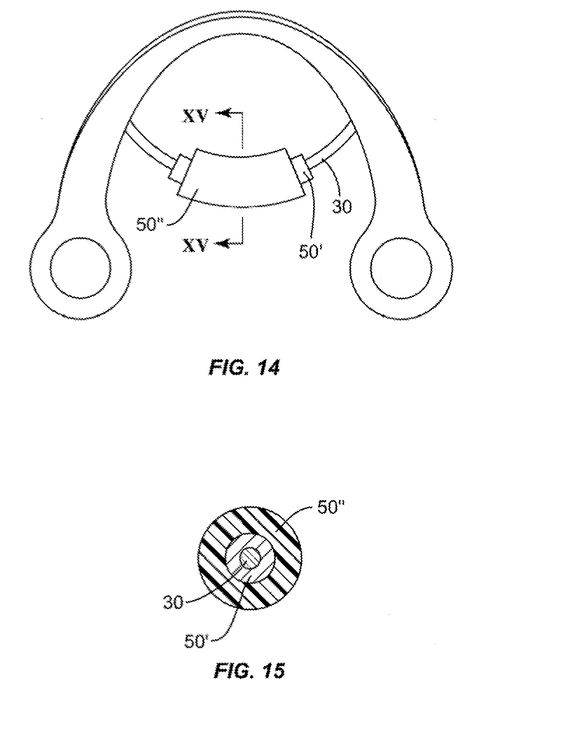

Bumper portion 50′ may be formed from a flexible, elastic or compressible material as a separate element which may be connected to support legs 20a′ and 20b′. See FIG. 6. Any suitable connection mechanism may be used to connect bumper portion 50′ to support legs 20a′ and 20b′. For example, a cross-bar 30 may extend between support leg 20a′ and support leg 20b′ and bumper portion 50′ may be placed over cross-bar 30. See e.g. FIGS. 6-8 and 11. In this configuration, bumper portion 50′ may be formed to have a generally tubular shape, which has a longitudinal axis that is straight or curved, depending on the configuration of cross-bar 30. Bumper portion 50′ defines a lumen therethrough and through which cross-bar 30 may extend to hold bumper portion 50′ in place. If desired, another bumper portion 50″ may be located over bumper 50′. See FIGS. 14 and 15. Cross-bar 30 may have a straight or curved configuration. In addition, cross-bar 30 need not extend completely across the gap between support leg 20a′ and support leg 20b′ but may instead only extend a short distance across the gap between support leg 20a′ and support leg 20b′. The extent that cross-bar 30 extends across the gap should be sufficient to support bumper portion 50′ and thus support the spinous process of the L5 vertebra during extension.

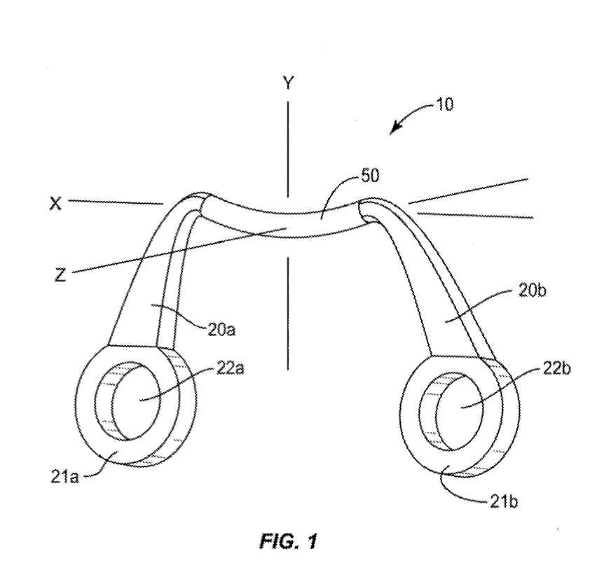

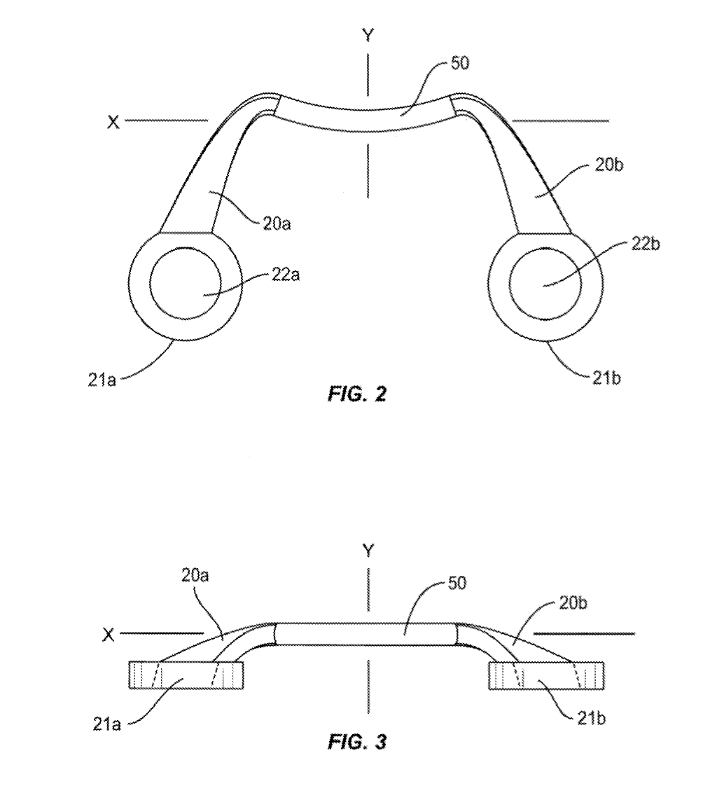

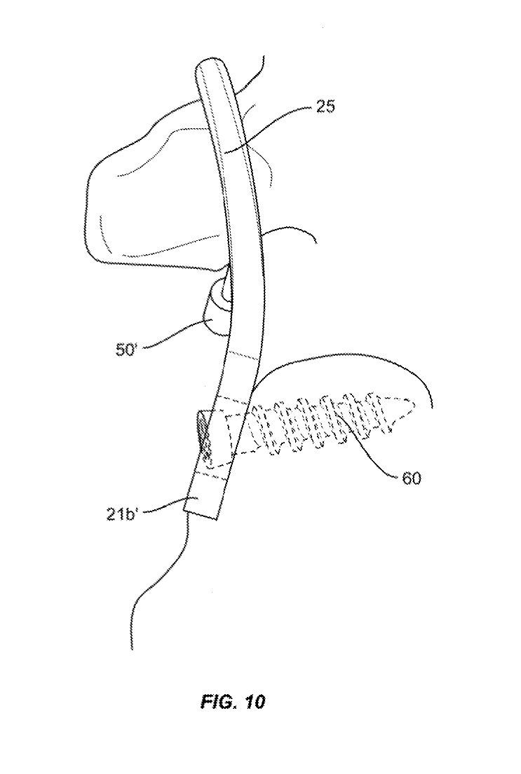

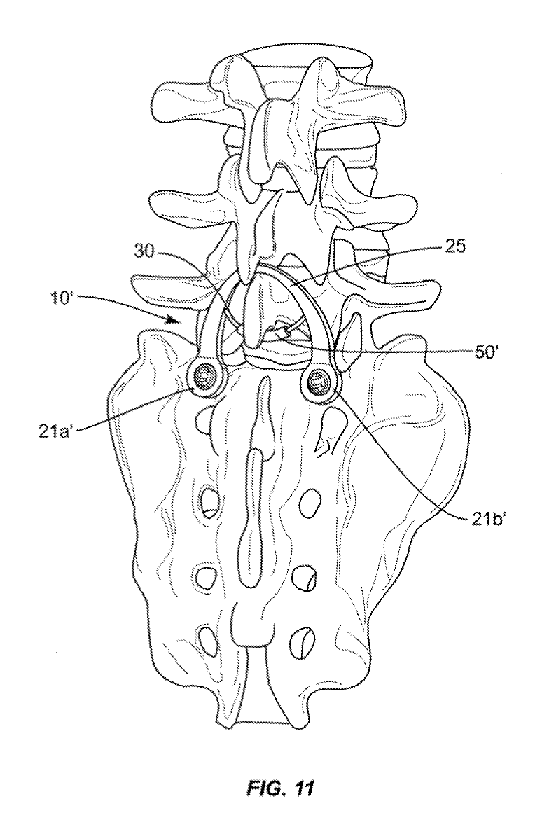

In the embodiment shown in FIGS. 1-5, support legs 20a and 20b and bumper 50 may be formed to provide a single, unitary configuration giving implant 10 a generally inverted U shape. In the embodiment shown in FIGS. 6-11, support legs 20a′ and 20b′ are formed as a single, unitary element with a superior portion 25 and cross-bar 30 giving implant 10′ more of an A shape. If desired, cross-bar may be formed with support legs 20a and 20b and superior portion 25 as a single, unitary element. In this embodiment, the superior portion 25 is adapted to extend over the superior portion of the L5 spinous process when implant 10′ is fixed in the desired location of the patient's anatomy. See FIGS. 10 and 11.

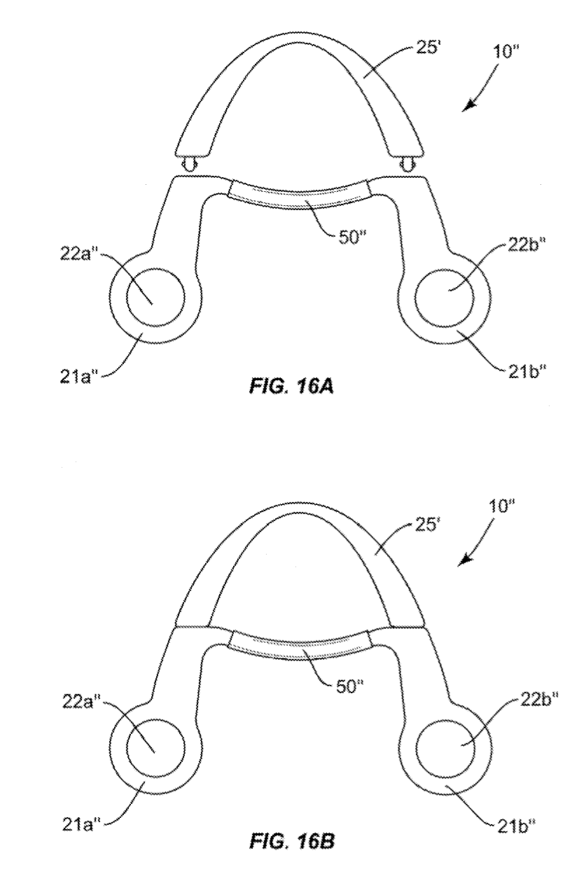

In a further embodiment, the superior portion 25′ of implant 10″ may be formed as a separate element that may be disconnected from the remainder of implant 10″. See FIGS. 16A and 16B. The inferior portion of implant 10″ may have a configuration similar to that shown in FIGS. 1-5 but with a mechanism to allow it to be connected to superior portion 25. For example, receiving slots may be formed in either the inferior portion of implant 10″ or superior portion 25′ that receive projections formed in the other of either superior portion 25′ or the inferior portion. Such a mechanism may include, but is not limited to, a mechanical detent mechanism. Alternatively, implant 10″ may have a configuration similar to the embodiment shown in FIGS. 6-11, but with a removable superior portion that may be locked in place. This further embodiment facilitates implantation of implant 10″ into a patient's anatomy because the inferior portion of implant 10″ can be fixed to the inferior vertebra first. Once that part of the procedure is accomplished and the inferior portion of implant 10″ is located in its desired position, superior portion 25′ can then be connected to the inferior portion and looped over the superior spinous process.

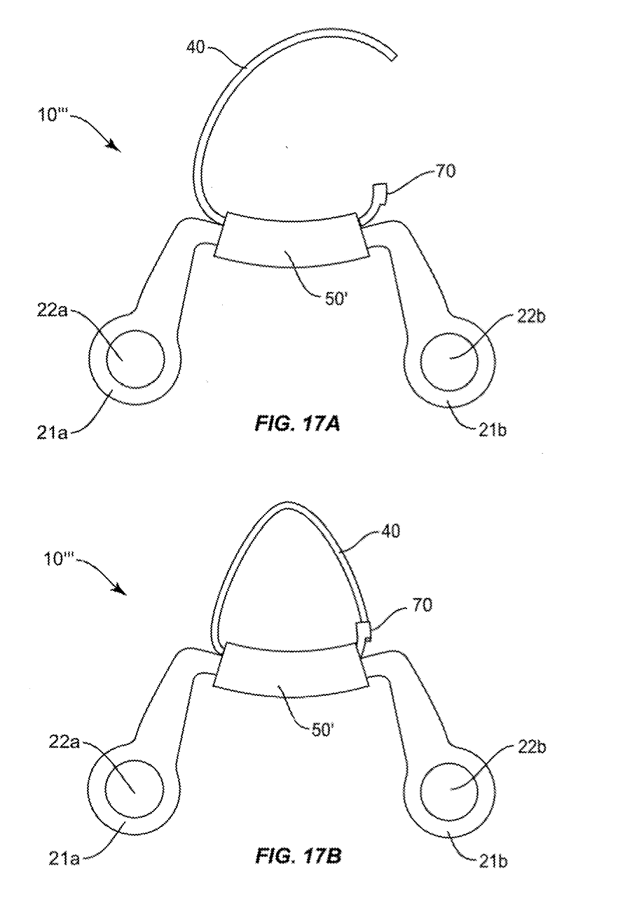

In a still further embodiment, implant 10′″ may include bumper portion 50′ as well as a separate tether 40 extending through the lumen of bumper 50. Tether 40 is adapted to extend over the superior portion of the superior spinous process. As shown in FIG. 17A, tether 40 may have two free ends which may be initially left unconnected to each other to allow tether 40 to be looped over the superior spinous process. Thereafter, as shown in FIG. 17B the free ends of tether 40 may be coupled together after implant 10 is properly fixed in place in the patient's anatomy. Tether 40 helps to maintain implant 10 in the proper position in the patient's anatomy during extension and flexion. Any suitable locking mechanism 70, such as a set screw, crimp, or ratchet, or even merely tying and knotting the free ends together, may be used to tighten and lock tether 40 around the spinous process of the L5 vertebra to the desired extent.

The overall shape of the spinal implant transfers load from the L5 spinous process to the S1 pedicles instead of to the S1 spinous process or the S1 laminae. This is especially helpful since the small size and shape of the S1 spinous process may not provide adequate support for an implant. The shape of the spinal implant also provides a better fit in the L5/S1 space and therefore offers greater stability. The spinal implant described herein provides clearance so that the bumper portion does not engage the inferior spinous process. This results in practically no load being transferred from the spinal implant to the inferior spinous process.

The support legs and the superior portion and cross-bar may be formed from any suitable biocompatible material including metal, such as titanium alloys, and plastic, such as PEEK or HDPE. The bumper may be formed from an elastic, flexible or compressible material such as a silicone elastomer having a durometer of between about 55A and about 85A.

While various embodiments of the spinal implant have been described above, it should be understood that they have been presented by way of example only, and not limitation. Many modifications and variations will be apparent to the practitioner skilled in the art. The foregoing description of the spinal implant is not intended to be exhaustive or to limit the scope of the invention. It is intended that the scope of the invention be defined by the following claims and their equivalents.

What is claimed is:

1. A device adapted to be disposed between two adjacent spinous processes, comprising:
a first support leg extending from a first fixation end;
a second support leg extending from a second fixation end;
a superior portion connecting the first support leg and the second support leg, the superior portion extending in a direction remote from the first fixation end and the second fixation end;
a bumper connected to the first support leg and the second support leg such that the bumper is disposed between, on one end the first fixation end and the second fixation end, and on another end the superior portion; and wherein
the first fixation end defines a first passage and the second fixation end defines a second passage and the superior portion is adapted to extend over a superior portion of a superior spinous process and the first passage and second passage are oriented at an angle of about 60 degrees away from the sagittal plane in a posterior to anterior direction and at an angle of about 5 degrees toward the superior portion in a posterior to anterior direction.

2. The device of claim 1 wherein the device defines a first medial portion adjacent to the first support leg and a second medial portion adjacent to the second support leg and further comprising a cross-bar extending between the first medial portion and the second medial portion.

3. The device of claim 2 wherein the bumper is disposed about the cross-bar.

4. The device of claim 2 wherein the bumper is more flexible than the first support leg and the second support leg.

5. The device of claim 4 wherein the first support leg, the second support leg, the superior portion and the cross-bar are formed as a unitary element.

6. The device of claim 4 wherein the superior portion is removably connected to the first support leg and the second support leg.

7. A device, comprising:
a first support leg having a first fixation end and a first superior end;
a second support leg having a second fixation end and a second superior end; and
a bumper adjacent to the first superior end and the second superior end wherein the bumper, the first support leg and the second support leg are formed as a single, unitary element formed from the same material.

8. The device of claim 7 wherein the first fixation end defines a first passage and the second fixation end defines a second passage and the first passage and second passage are oriented at an angle of about 60 degrees away from the sagittal plane in a posterior to anterior direction and at an angle of about 5 degrees toward a superior portion of the device in a posterior to anterior direction.

9. The device of claim 7 wherein the bumper is a cross-bar extending between the first superior end and the second superior end and further including a tubular element disposed over the bumper.

10. The device of claim 9 wherein the tubular element is more flexible than the first support leg and the second support leg.

11. The device of claim 7 further comprising a tether operatively connected to the device.

12. The device of claim 9 wherein the tether is connected to the tubular element.

13. The device of, claim 11 wherein the tether includes a first free end and a second free end and a lock adapted to join the first free end to the second free end.

14. The device of claim 7 wherein the first fixation end is oriented at a first angle to the first support leg and the second fixation end is oriented at a second angle to the second support leg.

15. The device of claim 14 wherein the first angle and the second angle are between about 20 degrees and about 40 degrees.

16. A device, comprising:
a unitary support rod extending from a first fixation end to a superior portion and then to a second fixation end; and wherein
the first fixation end defines a first passage and the second fixation end defines a second passage and the first passage and second passage are oriented at an angle of about 60 degrees away from the sagittal plane in a posterior to anterior direction and at an angle of about 5 degrees toward the superior portion in a posterior to anterior direction and the first fixation end extends from the support rod at a first angle and the second fixation end extends from the support rod at a second angle.

17. The device of claim 16 wherein the first angle and the second angle are between about 20 degrees and about 40 degrees.

18. The device of claim 16 further comprising a bumper extending between two portions of the support rod.

19. The device of claim 18 wherein the bumper defines the superior extent of the device.

20. The device of claim 18 wherein the bumper is located along a medial portion of the device.
